(19) United States
(12) Patent Application Publication
(10) Pub. No.: US 2005/0239719 A1
(43) Pub. Date: Oct. 27, 2005

(54) Methods of using and compositions comprising thalidomide for the treatment and management of pulmonary hypertension

(76) Inventor: Jerome B. Zeldis, Princeton, NJ (US)

Correspondence Address: JONES DAY, 222 EAST 41ST ST, NEW YORK, NY, 10017, US

(21) Appl. No.: 11/111,186

(22) Filed: Apr. 21, 2005

Related U.S. Application Data
(60) Provisional application No. 60/565,169, filed on Apr. 23, 2004.

(51) Int. Cl. A61K 31/704; A61K 31/454; A61K 31/455; A61K 31/554; A61K 31/519; A61K 31/366

(52) U.S. Cl. 514/26; 514/323; 514/460; 514/457; 514/252.16; 514/548; 514/211.07; 514/355

(57) ABSTRACT

Methods of treating, preventing and managing pulmonary hypertension are disclosed. Specific methods encompass the administration of thalidomide, or a pharmaceutically acceptable salt, solvate (e.g., hydrate), stereoisomer, clathrate, or prodrug thereof, alone or in combination with a second active agent, surgery and/or lung transplantation. Specific second active agents are capable of reducing pulmonary artery pressure. Pharmaceutical compositions, single unit dosage forms, and kits suitable for use in methods of the invention are also disclosed.

This application claims the benefit of U.S. provisional application No. 60/565,169, filed Apr. 23, 2004, the entirety of which is incorporated herein by reference.

### 1. FIELD OF THE INVENTION

This invention relates to methods of treating, preventing and managing pulmonary hypertension which comprise the administration of thalidomide alone or in combination with a known therapeutic. The invention also relates to pharmaceutical compositions and dosing regimens. In particular, the invention encompasses the use of thalidomide in conjunction with surgery, transplantation therapy and/or other standard therapies for pulmonary hypertension.

### 2. BACKGROUND OF THE INVENTION

### 2.1. Pathobiology of PH

Pulmonary hypertension (“PH”) refers to a disease characterized by sustained elevations of pulmonary artery pressure. L. J. Rubin, The New England Journal of Medicine, 336(2): 111, 1997. PH occurs from diverse etiologies, and thus a classification of the disease has been helpful. S. Rich, Advances in Pulmonary Hypertension, 1(1):3, 2002. World Health Organization (WHO) classified pulmonary hypertension into groups based on known causes, and defined primary pulmonary hypertension as a separate entity of unknown cause. Id. In addition, a functional classification of heart disease, patterned after the New York Heart Association (NYHA) Functional Classification for the disease, was developed by WHO to allow comparisons of patients with respect to the clinical severity of the disease. Id. The functional classifications are shown below in Table 1.

TABLE 1
WHO Functional Classification of Pulmonary Hypertension (PH)

| | |
|---|---|
| Class I | Patients with PH but without resulting limitation of |
| | physical activity. |
| Class II | Patients with PH resulting in slight limitation of |
| | physical activity. |
| Class III | Patients with PH resulting in marked limitation of |
| | physical activity. |
| Class IV | Patients with PH with inability to carry out any |
| | physical activity without symptoms. |

Pulmonary hypertension (PH) is divided into primary and secondary forms. S. Rich, Advances in Pulmonary Hypertension, 1(1):3, 2002. Primary pulmonary hypertension (PPH) is a disease of unknown etiology, whereas secondary pulmonary hypertension (SPH) is due to either intrinsic parenchymal disease of the lung or disease extrinsic to the lung. Id. PPH is classified into three histopathological patterns of plexogenic arteriopathy, recurrent thromboembolism, and veno-occlusive disease. Id. Patients with PPH are subcategorized into sporadic and familial. Id., p. 4. Reportedly about 12% of patients with PPH have familial PPH. Id. However, this may underestimate true familial PPH prevalence, because it can skip several generations. Id. It has been recently reported that the PPH-1 gene is present in approximately half the patients with familial PPH. Z. Deng, Am J Respir Crit Care Med., 161:1055-1059, 2000. Twenty-five percent of patients with sporadic PPH reportedly test positive for the PPH-1 gene. Id.

In SPH, the mechanisms are often multi-factorial, depending on the underlying etiology. S. Rich, Advances in Pulmonary Hypertension, 1(1):4, 2002. Cardiac disorders, pulmonary disorders and combinations thereof are the most common causes of SPH. Id. Patients with pulmonary arterial hypertension related to collagen vascular diseases have clinical features representing both entities. Id. It is most common for the collagen vascular disease to manifest itself years before the onset of PH, but on occasion the opposite has occurred. Id.

Congenital systemic to pulmonary shunts can cause PH that may be related to the increased blood flow and pressure transmitted to the pulmonary circulation. Id. The association between liver disease and PH appears to be related to portal hypertension. Id. Why portal hypertension leads to PH is not fully understood. Id.

The presence of the HIV virus can induce PH, probably through activation of cytokine or growth factor pathways. Id. Several drugs and toxins have also been associated with the development of PH, although a causal relationship with many is uncertain. Id. The strongest association between drug ingestion and the development of PH has been made with the fenfluramines. Id. Although the syndrome is indistinguishable from PPH, studies suggest that patients tend to have a more aggressive disease with a poorer prognosis than similar patients with PPH. Id. This may be a result of the fenfluramines triggering a unique molecular pathway that produces pulmonary vasculopathy. Id.

Persistent pulmonary hypertension of the newborn is distinguished from congenital abnormalities of the heart and pulmonary vasculature, is similar to PPH, and is typically somewhat more responsive to acute and chronic vasodilator therapies. S. Rich, Advances in Pulmonary Hypertension, 1(1):5, 2002.

In other patients, PH is caused by pulmonary venous hypertension that has a pathophysiology and clinical course that is markedly different from pulmonary arterial hypertension. Id. Orthopnea and paroxysmal nocturnal dyspnea are characteristic features, which may precede dyspnea. Id. These patients often have a history of chronic congestive heart failure and/or recurring pulmonary edema, which then becomes obscured when right ventricular failure ensues. Id.

PH is also associated with disorders of the respiratory system and/or hypoxemia, including chronic obstructive pulmonary disease, interstitial lung disease, sleep-disordered breathing, alveolar hypoventilation disorders, chronic exposure to high altitude, neonatal lung disease and alveolar-capillary dysplasia. Id. Although hypoxemia may coexist in all forms of PH, it is the hallmark of these conditions. Id. These patients are often dyspneic at rest as well as with minimal activity, with only subtle clinical features of PH. Id.

PH can result from chronic thrombotic or embolic diseases, such as sickle cell disease, other coagulation disorder, chronic thromboemboli, connective tissue disease, lupus, and schistosomiasis. S. Rich, Advances in Pulmonary Hypertension, 1(1):5-6, 2002. These patients often present with clinical signs and symptoms that are indistinguishable from pulmonary arterial hypertension. Id.

Inflammatory diseases such as schistosomiasis, sarcoidosis and pulmonary capillary hemangiomatosis directly affect the pulmonary vasculature, and can also result in PH. S. Rich, Advances in Pulmonary Hypertension, 1(1):6, 2002. Schistosomiasis is probably the most common cause of PH worldwide, although it is virtually never seen in Westernized countries. Id. Sarcoidosis can cause extensive destruction of the pulmonary parenchyma and pulmonary vascular bed, and can cause PH merely by lung destruction and resulting hypoxemia. Id. Patients may also develop PH presumably due to the involvement of the pulmonary circulation from the sarcoid process. Id. Pulmonary capillary hemangiomatosis is an extremely rare disorder involving the pulmonary capillary bed that can present in different stages. Id. It is often associated with frequent hemoptysis, severe PH, and a progressive fatal course in a short period of time. Id.

The common symptoms of PH reported in a national prospective study include dyspnea, fatigue, weakness, chest pain, recurrent syncope, seizures, light-headedness, neurologic deficits, leg edema and palpitations. Rich, Annals of Internal Medicine, 107; 217, 1987; The Merck Manual, 595 (17^{th} ed. 1999). Within the pulmonary arterioles, intimal hyperplasia and consequent narrowing of the vessel lumen are present in patients with PH. Id. Areas of medial (smooth muscle) hypertrophy and hyperplasia, irreversible plexiform lesions and necrotizing arteries occur in more advanced cases. Id.

The pathophysiology of PH is poorly understood. An insult to the endothelium such as hormonal or mechanical impact is thought to result in vascular scarring, endothelial dysfunction, and intimal and medial proliferation. The Merck Manual 1703 (17^{th} ed. 1999).

Loss of pulmonary vasodilators and an excess of vasoconstrictors may play a role in PH. Id. Increased expression of the potent vasoconstrictor endothelin-1 (ET-1) was found in the muscular pulmonary arteries and plexiform lesions of PH patients. R. N. Channick, Advances in Pulmonary Hypertension, 1(1):14, 2002. Moreover, pulmonary arteries in the lungs of patients with PH reportedly have decreased expressions of prostacyclin (PGI₂) synthase and endothelial cell nitric oxide synthase (eNOS). L. J. Rubin, Clinics in Chest Medicine, 22(3): 2001. The reduced expressions are believed to key alterations of the pulmonary endothelium in severe PH. Id. Decreased levels of PGI₂ and nitric oxide (NO) may be causally linked to increased pulmonary vasoconstriction, as well as more advanced structural alterations of pulmonary arteries, growth of vascular smooth muscle cell, and increased endothelial cell apoptosis secondary to loss of NO-protective effects on endothelial cells. Id. These effects may be of importance in pathogenesis and progression of PH. Id.

A recent study of PH proposed that dysfunctional endothelial cells have a central role in the initiation and progression of PH. L. J. Rubin, Clinics in Chest Medicine, 22(3), 2001. It was demonstrated that overgrown endothelial cells in severe PH obliterate the vascular lumen and contribute to the disruption of pulmonary flow, which may suggest that somatic mutations of angiogenesis- or apotosis-related genes such as transforming growth factor-beta (TGF-beta) receptor 2 may underlie the proliferation of endothelial cells in PPH patients. Id. The loss of these important cell growth mechanisms allows for the clonal expansion of endothelial cells from a single cell that has acquired a selective growth advantage. Id. On the other hand, the proliferated endothelial cells in SPH patients are believed polygonal. Id. It follows from this finding that local vascular factors such as increased shear stress, rather than mutations, play major roles in triggering endothelial cell proliferation. Id. In PPH and SPH, it is postulated that the pulmonary vascular bed contains progenitor-like cells with the capacity of dysregulated growth. Id. The main difference in the pathogenesis of primary and secondary pulmonary endothelial cell proliferation therefore may be the initial mechanism involved in the recruitment of endothelial progenitor-like cell. Id. In PPH, the proliferation of endothelial cells occurs from a mutated single cell, whereas in SPH, several progenitor-like cells are activated. Id.

### 2.2. PH Treatments

Current treatment of PH depends on the stage and the mechanism of the disease. Typical treatments for PH include anticoagulation, oxygen supplementation, conventional vasodilator therapy, transplantation and surgical care.

Several studies suggest that survival is increased when the patient is treated with anticoagulant therapy, regardless of histopathologic subtype. Rubin et al., The New England Journal of Medicine, 336(2); 115, 1997. Warfarin is used to maintain an International Normalized Ratio of 1.5- to 2-times the control value, provided no contraindication to anticoagulation is present. V. F. Tapson, Advances in Pulmonary Hypertension, 1(1): 16, 2002.

Digoxin is used to prevent and treat supraventricular arrhythmias associated with SPH and for patients who have concomitant left heart failure. However, no randomized controlled clinical study has been performed to validate this strategy for patients with PPH. V. F. Tapson, Advances in Pulmonary Hypertension, 1(1): 16, 2002. Diuretics are reportedly useful in reducing excessive preload in patients with right heart failure. Rubin et al., The New England Journal of Medicine, 336(2); 115, 1997. Oxygen supplementation is used in those patients with resting or exercise-induced hypoxemia. Id. and V. F. Tapson, Advances in Pulmonary Hypertension, 1(1):16, 2002.

Arterial septostomy or lung transplant is indicated for patients who do not respond to medical therapy. The Merck Manual 1704 (17^{th} ed. 1999); L. J. Rubin, Advances in Pulmonary Hypertension, 1(1): 16 and 19, 2002. Arterial septostomy is intended to serve as a bridge to transplantation. Id. However, very few have extensive experience with arterial septostomy. Id. The availability of lung organ for transplantation is also limited. Id. at 19. Further, long-term complications after transplantation, such as chronic rejection and opportunistic infections, have hampered its long-term efficacy in many patients. Id.

Medications presently used for the treatment of PH include calcium channel blockers and pulmonary vasodilators. The Merck Manual 1704 (17^{th} ed. 1999); V. F. Tapson, Advances in Pulmonary Hypertension, 1(1): 16, 2002. Calcium channel blockers are the most widely used class of drugs for PH. Studies suggest that the drugs produce improvements in 20-30% of PPH patients. The New England Journal of Medicine, 336(2); 114, 1997.

The currently available vasodilators are epoprostenol (EPO, Floran®), treprostinil (Remodulin®) and bosentan (Tracleer®). V. F. Tapson, Advances in Pulmonary Hypertension, 1(1): 16, 2002; R. N. Channick, Advances in Pulmonary Hypertension, 1(1): 14-15, 2002. Recently, bosentan has been approved for initial PH therapy in patients with NYHA class III and IV symptoms. This endothelially active agent reportedly improves exercise capacity and shows promise in halting or reversing pulmonary vascular insult. However, the usefulness of vasodilator therapy is controversial in patients who have an acute reduction in vascular resistance resulting from an increased cardiac output without a fall in pulmonary artery pressure. Rubin et al., The New England Journal of Medicine, 336(2); 114, 1997. Therefore, a need remains for safe and effective methods of treating and managing PH.

### 2.3. Thalidomide

Thalidomide is a racemic compound sold under the tradename Thalomid® and chemically named α-(N-phthalimido)glutarimide or 2-(2,6-dioxo-3-piperidinyl)-1H-isoindole-1,3(2H)-dione. The compound has structure I:

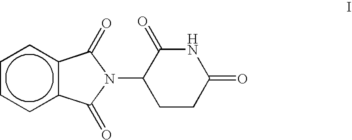

Thalidomide was originally developed in the 1950's to treat morning sickness, but due to its teratogenic effects was withdrawn from use. Thalidomide has been approved in the United States for the acute treatment of the cutaneous manifestations of erythema nodosum leprosum in leprosy. Physicians' Desk Reference, 1153-1157 (57th ed., 2003). Because its administration to pregnant women can cause birth defects, the sale of thalidomide is strictly controlled. Id. Thalidomide has reportedly been studied in the treatment of other diseases, such as chronic graft-vs-host disease, rheumatoid arthritis, sarcoidosis, several inflammatory skin diseases, and inflammatory bowel disease. See generally, Koch, H. P., Prog. Med. Chem. 22:165-242 (1985). See also, Moller, D. R., et al., J. Immunol. 159:5157-5161 (1997); Vasiliauskas, E. A., etal., Gastroenterology 117:1278-1287 (1999); Ehrenpreis, E. D., et al., Gastroenterology 117:1271-1277 (1999). It has further been alleged that thalidomide can be combined with other drugs to treat ischemia/repercussion associated with coronary and cerebral occlusion. See U.S. Pat. No. 5,643,915, which is incorporated herein by reference.

Thalidomide has reportedly been clinically investigated in the treatment of specific types of cancers, such as refractory multiple myeloma, brain, melanoma, breast, colon, mesothelioma, and renal cell carcinoma. See, e.g., Singhal, S., et al., New England J. Med. 341(21):1565-1571 (1999); and Marx, G. M., et al., Proc. Am. Soc. Clin. Oncology 18:454a (1999). It has further been reported that thalidomide can be used to prevent the development of chronic cardiomyopathy in rats caused by doxorubicin. Costa, P. T., et al., Blood 92(10:suppl. 1):235b (1998). Other reports concerning the use of thalidomide in the treatment of specific cancers include its combination with carboplatin in the treatment of glioblastoma multiforme. McCann, J., Drug Topics 41-42 (Jun. 21, 1999). Thalidomide has also reportedly been used as an antiemetic during the treatment of astrocytoma. Zwart, D., Arzneim.-Forsch. 16(12):1688-1689 (1966).

If there is a general mechanism by which thalidomide aids in the treatment of some cancers, its nature remains unclear. See, e.g., Moreira, A. L., et al., J. Expr. Med. 177:1675-1680 (1993); McHugh, S. M., et al., Clin. Exper. Immunol. 99:160-167 (1995); and Moller, D. R., et al., J. Immunol. 159:5157-5161 (1997). It has been reported, however, that thalidomide is an antiangiogenic agent that can suppress tumor necrosis factor α (TNF-α) and interleukin 12 (IL-12) production. See, e.g., Moller, D. R., et al., J. Immunol. 159:5157-5161 (1997); Moreira, A. L., et al., J. Exp. Med. 177:1675-1680 (1993); U.S. Pat. Nos. 5,593,990, 5,629,327, and 5,712,291 to D'Amato and U.S. Pat. No. 5,385,901 to Kaplan. And in vitro studies suggest that thalidomide affects the production of a variety of other proteins. See, e.g., McHugh, S. M., et al., Clin. Exp. Immunol. 99:160-167 (1995). Thalidomide may also affect mechanisms related to epithelial or endothelial function or growth. D'Amato M., et al., Proc. Natl. Acad. Sci. 91:4082-4085 (1994).

### 3. SUMMARY OF THE INVENTION

This invention encompasses methods of treating or preventing pulmonary hypertension (“PH”) which comprise administering to a patient in need thereof a therapeutically or prophylactically effective amount of thalidomide or a pharmaceutically acceptable salt, solvate (e.g., hydrate), stereoisomer, clathrate, or prodrug thereof. The invention also encompasses methods of managing PH (e.g., lengthening the time of remission) which comprise administering to a patient in need of such management a therapeutically or prophylactically effective amount of thalidomide or a pharmaceutically acceptable salt, solvate (e.g., hydrate), stereoisomer, clathrate, or prodrug thereof.

One embodiment of the invention encompasses the use of thalidomide alone or in combination with conventional therapeutics presently used to treat, prevent or manage PH such as, but not limited to, anticoagulants, diuretics, cardiac glycosides, calcium channel blockers, vasodilators, prostacyclin analogues, endothelin antagonists, phosphodiesterase inhibitors, endopeptidase inhibitors, lipid lowering agents, thromboxane inhibitors, surgery and lung transplantations.

The invention further encompasses pharmaceutical compositions, single unit dosage forms, and kits suitable for use in treating, preventing and/or managing PH, which comprise thalidomide, or a pharmaceutically acceptable salt, solvate (e.g., hydrate), stereoisomer, clathrate, or prodrug thereof, and an optional second agent.

### 4. DETAILED DESCRIPTION OF THE INVENTION

A first embodiment of the invention encompasses methods of treating, preventing or managing PH which comprise administering to a patient in need of such treatment, prevention or management a therapeutically or prophylactically effective amount of thalidomide, or a pharmaceutically acceptable salt, solvate (e.g., hydrate), stereoisomer, clathrate, or prodrug thereof.

As used herein, and unless otherwise indicated, the terms “pulmonary hypertension,” “PH” and “PH and related disorders” include, but are not limited to: primary pulmonary hypertension (PPH); secondary pulmonary hypertension (SPH); familial PPH; sporadic PPH; precapillary pulmonary hypertension; pulmonary arterial hypertension (PAH); pulmonary artery hypertension; idiopathic pulmonary hypertension; thrombotic pulmonary arteriopathy (TPA); plexogenic pulmonary arteriopathy; functional classes I to IV pulmonary hypertension; and pulmonary hypertension associated with, related to, or secondary to, left ventricular dysfunction, mitral valvular disease, constrictive pericarditis, aortic stenosis, cardiomyopathy, mediastinal fibrosis, anomalous pulmonary venous drainage, pulmonary venoocclusive disease, collagen vasular disease, congenital heart disease, HIV virus infection, drugs and toxins such as fenfluramines, congenital heart disease, pulmonary venous hypertension, chronic obstructive pulmonary disease, interstitial lung disease, sleep-disordered breathing, alveolar hypoventilation disorder, chronic exposure to high altitude, neonatal lung disease, alveolar-capillary dysplasia, sickle cell disease, other coagulation disorder, chronic thromboemboli, connective tissue disease, lupus, schistosomiasis, sarcoidosis or pulmonary capillary hemangiomatosis.

Another embodiment of the invention encompasses a method of treating, preventing and/or managing PH, which comprises administering to a patient in need of such treatment, prevention and/or management a therapeutically or prophylactically effective amount of thalidomide, or a pharmaceutically acceptable salt, solvate (e.g., hydrate), stereoisomer, clathrate, or prodrug thereof, and a therapeutically or prophylactically effective amount of a second active agent.

Examples of second active agents include, but are not limited to, anticoagulants, diuretics, cardiac glycosides, calcium channel blockers, vasodilators, prostacyclin analogues, endothelin antagonists, phosphodiesterase inhibitors, endopeptidase inhibitors, lipid lowering agents, thromboxane inhibitors, or other agents found, for example, in the Physician's Desk Reference 2003. Second active agents can be large molecules (e.g., proteins) or small molecules (e.g., synthetic inorganic, organometallic, or organic molecules). Examples of specific second active agents include, but are not limited to, amlodipine, diltiazem, nifedipine, adenosine, epoprostenol (Floran®), treprostinil (Remodulin®), bosentan (Tracleer®), warfarin, digoxin, nitric oxide, L-arginine, iloprost, betaprost, and sildenafil (Viagra®).

Another embodiment of the invention encompasses a method of reversing, reducing or avoiding an adverse effect associated with the administration of a therapeutic used to treat PH, which comprises administering to a patient in need thereof a therapeutically or prophylactically effective amount of thalidomide, or a pharmaceutically acceptable salt, solvate (e.g., hydrate), stereoisomer, clathrate, or prodrug thereof, and an optional second active agent.

Procedures such as lung transplantation may be necessary to treat PH patients who have failed to respond to medical therapy. It is believed that the combined use of thalidomide and lung transplantation in a patient suffering from PH can be particularly beneficial. It is believed that thalidomide can work in combination with transplantation therapy, reducing complications such as chronic rejection and opportunistic infections associated with the transplantation. Therefore, this invention encompasses a method of treating or managing PH, which comprises administering to a patient (e.g., a human) thalidomide, or a pharmaceutically acceptable salt, solvate (e.g., hydrate), stereoisomer, clathrate, or prodrug thereof, before, during, or after transplantation therapy.

Another embodiment of the invention encompasses pharmaceutical compositions that can be used in methods of the invention. Specific compositions comprise thalidomide, or a pharmaceutically acceptable salt, solvate (e.g., hydrate), stereoisomer, clathrate, or prodrug thereof, and an optional second active agent.

Also encompassed by the invention are single unit dosage forms comprising thalidomide, or a pharmaceutically acceptable salt, solvate (e.g., hydrate), stereoisomer, clathrate, or prodrug thereof.

The invention also encompasses kits which comprise thalidomide, or a pharmaceutically acceptable salt, solvate (e.g., hydrate), stereoisomer, clathrate, or prodrug thereof, and a second active agent. For example, a kit may contain the compound of the invention, and calcium channel blockers, vasodilators, prostacyclin analogues, endothelin antagonists, phosphodiesterase inhibitors, endopeptidase inhibitors, lipid lowering agents, thromboxane inhibitors or other agents used to treat PH patients.

### 4.1. Compounds of the Invention

Compounds used in the invention include racemic thalidomide, stereomerically enriched or stereomerically pure thalidomide, and a pharmaceutically acceptable salt, solvate (e.g., hydrate), stereoisomer, clathrate, and prodrug thereof.

As used herein and unless otherwise indicated, the term “stereomerically pure” means a composition that comprises one stereoisomer of a compound and is substantially free of other stereoisomer of that compound. A typical stereomerically pure compound comprises greater than about 80% by weight of one stereoisomer of the compound and less than about 20% by weight of other stereoisomer of the compound, more preferably greater than about 90% by weight of one stereoisomer of the compound and less than about 10% by weight of the other stereoisomer of the compound, even more preferably greater than about 95% by weight of one stereoisomer of the compound and less than about 5% by weight of the other stereoisomer of the compound, and most preferably greater than about 97% by weight of one stereoisomer of the compound and less than about 3% by weight of the other stereoisomer of the compound.

As used herein and unless otherwise indicated, the term “stereomerically enriched” means a composition that comprises greater than about 60% by weight of one stereoisomer of a compound, preferably greater than about 70% by weight, more preferably greater than about 80% by weight of one stereoisomer of the compound.

As used herein and unless otherwise indicated, the term “enantiomerically pure” means a stereomerically pure composition of a compound having one chiral center. Similarly, the term “enantiomerically enriched” means a stereomerically enriched composition of a compound having one chiral center.

Thalidomide can either be commercially purchased (from Celgene Corp., New Jersey) or prepared according to the known methods. See, e.g., I. D. Fratta et al., Toxicol. Appl. Pharmacol. 7, 268 (1965), and the references disclosed therein. Enantiomerically pure thalidomide can be resolved using known resolving agents or chiral columns as well as other standard synthetic organic chemistry techniques. See, e.g., Blaschke, Arzneimittelforschung 29: 1640-1642 (1979); Shealy et al., Chem. Indus. 1030 (1965); and Casini et al., Farmaco Ed. Sci. 19:563 (1964).

As used herein, unless otherwise specified, the term “pharmaceutically acceptable salt(s)” includes salts of acidic or basic moieties of the compound(s) to which the term refers. Basic moieties are capable of forming a wide variety of salts with various inorganic and organic acids. The acids that can be used to prepare pharmaceutically acceptable acid addition salts of such basic compounds are those that form non-toxic acid addition salts, i.e., salts containing pharmacologically acceptable anions. Suitable organic acids include, but are not limited to, maleic, fumaric, benzoic, ascorbic, succinic, acetic, formic, oxalic, propionic, tartaric, salicylic, citric, gluconic, lactic, mandelic, cinnamic, oleic, tannic, aspartic, stearic, palmitic, glycolic, glutamic, gluconic, glucaronic, saccharic, isonicotinic, methanesulfonic, ethanesulfonic, p-toluenesulfonic, benzenesulfonic acids, or pamoic (i.e., 1,1′-methylene-bis-(2-hydroxy-3-naphthoate) acids. Suitable inorganic acids include, but are not limited to, hydrochloric, hydrobromic, hydroiodic, sulfuric, phosphoric, or nitric acids. Compounds that include an amine moiety can form pharmaceutically acceptable salts with various amino acids, in addition to the acids mentioned above.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein to describe a compound or chemical moiety, the term “derivative” means a compound or chemical moiety wherein the degree of saturation of at least one bond has been changed (e.g., a single bond has been changed to a double or triple bond) or wherein at least one hydrogen atom is replaced with a different atom or a chemical moiety. Examples of different atoms and chemical moieties include, but are not limited to, halogen, oxygen, nitrogen, sulfur, hydroxy, methoxy, alkyl, amine, amide, ketone, and aldehyde.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of thalidomide that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of thalidomide that include —NO, —NO₂, —ONO, or —ONO₂ moieties.

As used herein and unless otherwise indicated, the terms “biohydrolyzable carbamate,” “biohydrolyzable carbonate,” “biohydrolyzable ureide,” “biohydrolyzable phosphate” mean a carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein and unless otherwise indicated, the term “biohydrolyzable ester” means an ester of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, alkoxyacyloxy esters, alkyl acylamino alkyl esters, and choline esters.

As used herein and unless otherwise indicated, the term “biohydrolyzable amide” means an amide of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable amides include, but are not limited to, lower alkyl amides, α-amino acid amides, alkoxyacyl amides, and alkylaminoalkylcarbonyl amides.

### 4.2. Second Active Agents

Thalidomide can be combined with other pharmacologically active compounds (“second active agents”) in methods and compositions of the invention. In a preferred embodiment, the second active agents are capable of reducing pulmonary artery pressure or vascular resistance, inhibiting thrombosis or thromboembolism, or ensuring compliance of patients. Examples of the second active agents include, but are not limited to, anticoagulants, diuretics, cardiac glycosides, calcium channel blockers, vasodilators, prostacyclin analogues, endothelin antagonists, phosphodiesterase inhibitors (e.g., PDE V inhibitors), endopeptidase inhibitors, lipid lowering agents, thromboxane inhibitors, and other therapeutics known to reduce pulmonary artery pressure.

Specific second active agents are anticoagulants, which are useful in the treatment of patients with PH who have an increased risk of thrombosis and thromboembolism. A particular anticoagulant is warfarin (Coumadin®).

Other second active agents include diuretics, cardiac glycosides, and oxygen. Digoxin therapy is used to improve right ventricular function in patients with right ventricular failure. Diuretics can be used to manage peripheral edema. Oxygen supplementation may be used in those patients with resting or exercise-induced hypoxemia.

Calcium channel blockers such as diltiazem and nifedipine can also be used as second active agents, particularly for vasoreactive patients at right heart catheterization. These drugs are thought to act on the vascular smooth muscle to dilate the pulmonary resistance vessels and lower the pulmonary artery pressure. V. F. Tapson, Advances in Pulmonary Hypertension, 1(1): 16-17, 2002.

Other second active agents include vasodilators, particularly for NYHA types III and IV patients with right heart failure who do not respond to calcium channel blockers or are unable to tolerate them. Examples of vasodilators include, but are not limited to, prostacyclin (e.g., prostaglandin I₂ (PGI₂), epoprostenol (EPO, Floran®), treprostinil (Remodulin®), and nitric oxide (NO).

Still other second active agents are endothelin antagonists. One example is bosentan (Tracleer®), which competitively binds to endothelin-1 (ET-1) receptors, causing reduction in pulmonary artery pressure.

Specific second active agents used in the invention include, but are not limited to, amlodipine, nifedipine, diltiazem, epoprostenol (Floran®), treprostinil (Remodulin®), bosentan (Tracleer®), prostacyclin, warfarin (Coumadin®), tadalafil (Cialis®), simvastatin (Zocor®), omapatrilat (Vanlev®), irbesartan (Avapro®), pravastatin (Pravachol®), digoxin, nitric oxide, L-arginine, iloprost, betaprost, and sildenafil (Viagra®).

### 4.3. Methods of Treatment and Management

Methods of this invention encompass methods of preventing, treating and/or managing various types of PH. As used herein, unless otherwise specified, the term “preventing” or “prophylaxis” includes, but is not limited to, inhibiting or averting one or more symptoms associated with PH. Symptoms associated with PH include, but are not limited to, dyspnea, fatigue, weakness, chest pain, recurrent syncope, seizures, light-headedness, neurologic deficits, leg edema and palpitations. As used herein, unless otherwise specified, the term “treating” refers to the administration of a composition after the onset of symptoms of PH, whereas “preventing” refers to the administration prior to the onset of symptoms, particularly to patients at risk of PH. As used herein and unless otherwise indicated, the term “managing” encompasses preventing the recurrence of PH in a patient who had suffered from PH, and/or lengthening the time that a patient who had suffered from PH remains in remission.

The invention encompasses methods of treating or managing patients who have been previously treated for PH, as well as those who have not previously been treated for PH. Because patients with PH have heterogenous clinical manifestations and varying clinical outcomes, it is preferred that patients should be treated according to the severity and stage of the disease. Methods and compositions of this invention can be used in various stages or types of PH including, but not limited to, primary PH, secondary PH and NYHA or WHO functional classes I to IV patients.

Methods encompassed by this invention comprise administering thalidomide, or a pharmaceutically acceptable salt, solvate (e.g., hydrate), stereoisomer, clathrate, or prodrug thereof to a patient (e.g., a human) suffering, or likely to suffer, from PH. Specific patient populations include young women, as PH affects mostly young reproductive-aged women. However, it is also common in women in their fifth and sixth decades of life. Patients with familial history of PH are also preferred candidates for preventive regimens.

In one embodiment of the invention, thalidomide is administered in a single or divided daily doses in an amount of from about 50 to about 2,000 mg/day, from about 100 to about 1,500 mg/day, from about 120 to about 1,200 mg/day, from about 150 to about 1,000 mg/day, or from about 200 to about 800 mg/day.

### 4.3.1 Combination Therapy With A Second Active Agent

Particular methods of the invention comprise administering 1) thalidomide, or a pharmaceutically acceptable salt, solvate (e.g., hydrate), stereoisomer, clathrate, or prodrug thereof, and 2) a second active agent. Examples of second active agents are also disclosed herein (see, e.g., section 4.2).

Administration of thalidomide and a second active agent to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. A preferred route of administration for thalidomide is oral. Another preferred route of administration for thalidomide is parenteral, particularly for patients who are in a peri-transplant period or in an end stage of PH. Preferred routes of administration for the second active agent of the invention are known to those of ordinary skill in the art such as in Physicians' Desk Reference (57^{th} ed., 2003).

The specific amount of the second active agent will depend on the specific agent used, the type of PH being treated or managed, the severity and stage of PH, and the amount(s) of thalidomide and any optional additional active agents concurrently administered to the patient. In specific embodiments of the invention, the second active agent is amlodipine, diltiazem, nifedipine, prostacyclin, epoprostenol (Floran®), treprostinil (Remodulin®), bosentan (Tracleer®), warfarin (Coumadin®), tadalafil (Cialis®), simvastatin (Zocor®), omapatrilat (Vanlev®), irbesartan (Avapro®), pravastatin (Pravachol®), digoxin, nitric oxide, L-arginine, iloprost, betaprost, or sildenafil (Viagra®).

In one embodiment of the invention, thalidomide is administered to reduce a period of treatment with a second active agent typically used to treat PH. In a particular embodiment, at the beginning of week one, from about 200 to about 800 mg/day of thalidomide is administered along with a second active agent in an amount that those of ordinary skill in the art can determine by their professional judgment. At the beginning of weeks 5, 9, 13, and 17, withdrawal of the second active agent may occur in increments of 25% of the initial dose of the second active agent. At the beginning of week 17, dose of the second active agent may be 0 mg/day if symptoms of a patient do not worsen. If symptoms of a patient worsen, dose of the second active agent may be increased to stabilize the patient.

In one embodiment of the invention, the second active agent is administered parenterally, orally or by inhalation. For example, epoprostenol (Floran®) is administered by continuous IV infusion via permanent indwelling central venous catheter. The initial dose of the drug is about 2-4 ng/kg/min, depending on initial response under close observation in the ICU with right heart flotation catheter in place. Subsequently, the dose is titrated based on follow-up outpatient evaluation and can exceed 40 ng/kg/min after one year of therapy in some patients. Iloprost is preferably administered by inhalation. Betaprost is preferably administered orally.

In another embodiment of the invention, treprostinil (Remodulin®) is administered by continuous subcutaneous infusion with an initial dose of about 1.25 ng/kg/min. The subsequent dose may be increased by about 1.25 ng/kg/min each week for four weeks, and then by 2.5 ng/kg/min each week. Preferably, the dose does not exceed about 40 ng/kg/min.

In another embodiment of the invention, bosentan (Tracleer®) is administered orally with a starting dose of about 62.5 mg twice a day for four weeks, followed by about 125 mg twice a day.

### 4.3.2 Use With Surgery or Transplantation

This invention encompasses a method of treating or managing PH, which comprises administering thalidomide, or a pharmaceutically acceptable salt, solvate (e.g., hydrate), stereoisomer, clathrate, or prodrug thereof, in conjunction with surgery or transplantation therapy. As discussed elsewhere herein, the treatment of PH varies, depending on the stage and mechanism of the disease. Arterial septostomy or lung transplantation may be necessary for PH patients who have failed to respond to medicinal therapy. The combined use of thalidomide and an arterial septostomy or lung transplantation is believed to be unexpectedly beneficial. Moreover, thalidomide exhibit immunomodulatory activities that may provide additive or synergistic effects when given before, concurrently with, or after surgery or transplantation therapy in patients with PH. For example, thalidomide can reduce complications associated with conventional therapies.

### 4.4. Pharmaceutical Compositions and Single Unit Dosage Forms

Pharmaceutical compositions can be used in the preparation of individual, single unit dosage forms. Pharmaceutical compositions and dosage forms of the invention comprise thalidomide, or a pharmaceutically acceptable salt, solvate (e.g., hydrate), stereoisomer, clathrate, or prodrug thereof. Pharmaceutical compositions and dosage forms of the invention can further comprise one or more excipients.

Pharmaceutical compositions and dosage forms of the invention can also comprise one or more additional active agents. Consequently, pharmaceutical compositions and dosage forms of the invention comprise the active agents disclosed herein (e.g., thalidomide, or a pharmaceutically acceptable salt, solvate (e.g., hydrate), stereoisomer, clathrate, or prodrug thereof, and a second active agent). Examples of optional additional active agents are disclosed herein (see, e.g., section 4.2).

Single unit dosage forms of the invention are suitable for oral, mucosal (e.g., nasal, sublingual, vaginal, buccal, or rectal), or parenteral (e.g., subcutaneous, intravenous, bolus injection, intramuscular, or intraarterial), transdermal or transcutaneous administration to a patent. Examples of dosage forms include, but are not limited to: tablets such as rapidly dissolving tablets; caplets; capsules, such as soft elastic gelatin capsules; cachets; troches; lozenges; tapes such as rapidly dissolving tapes in oral fluids; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active agents it comprises than a dosage form used in the chronic treatment of the same disease. Similarly, a parenteral dosage form may contain smaller amounts of one or more of the active agents it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active agents in the dosage form. For example, the decomposition of some active agents may be accelerated by some excipients such as lactose, or when exposed to water. Active agents that comprise primary or secondary amines are particularly susceptible to such accelerated decomposition. Consequently, this invention encompasses pharmaceutical compositions and dosage forms that contain little, if any, lactose other mono- or di-saccharides. As used herein, the term “lactose-free” means that the amount of lactose present, if any, is insufficient to substantially increase the degradation rate of an active agent.

Lactose-free compositions of the invention can comprise excipients that are well known in the art and are listed, for example, in the U.S. Pharmacopeia (USP) 25-NF20 (2002). In general, lactose-free compositions comprise active agents, a binder/filler, and a lubricant in pharmaceutically compatible and pharmaceutically acceptable amounts. Preferred lactose-free dosage forms comprise active agents, microcrystalline cellulose, pre-gelatinized starch, and magnesium stearate.

This invention further encompasses anhydrous pharmaceutical compositions and dosage forms comprising active agents, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing agents and low moisture or low humidity conditions. Pharmaceutical compositions and dosage forms that comprise lactose and at least one active agent that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active agent will decompose. Such compounds, which are referred to herein as “stabilizers,” include, but are not limited to, antioxidants such as ascorbic acid, pH buffers, or salt buffers.

Like the amounts and types of excipients, the amounts and specific types of active agents in a dosage form may differ depending on factors such as, but not limited to, the route by which it is to be administered to patients. However, typical dosage forms of the invention comprise thalidomide, or a pharmaceutically acceptable salt, solvate (e.g., hydrate), stereoisomer, clathrate, or prodrug thereof in an amount of from about 50 to about 2,000 mg. Typical dosage forms comprise thalidomide, or a pharmaceutically acceptable salt, solvate (e.g., hydrate), stereoisomer, clathrate, or prodrug thereof in an amount of about 50, 100, 200, 300 or 400 mg. Certain dosage forms further comprise a second active agent, for example, in an amount of from about 1 to about 1000 mg, from about 5 to about 500 mg, from about 10 to about 350 mg, or from about 50 to about 200 mg. Of course, the specific amount of second active agent will depend on the specific agent used, the type of PH being treated or managed, and the amount(s) of thalidomide, and any optional additional active agents concurrently administered to the patient.

### 4.4.1 Oral Dosage Forms

Pharmaceutical compositions of the invention that are suitable for oral administration can be presented as discrete dosage forms, such as, but are not limited to, tablets (e.g., chewable tablets and rapidly dissolving tablets), caplets, capsules (e.g., soft elastic gelatin capsules), liquids (e.g., flavored syrups), and tapes (e.g., rapidly dissolving tapes). Such dosage forms contain predetermined amounts of active agents, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active agents in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms. In general, pharmaceutical compositions and dosage forms are prepared by uniformly and intimately admixing the active agents with liquid carriers, finely divided solid carriers, or both, and then shaping the product into the desired presentation if necessary. If desired, dosage forms can be coated by standard aqueous or nonaqueous techniques.

For example, a tablet can be prepared by compression or molding. Compressed tablets can be prepared by compressing in a suitable machine the active agents in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active agents should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

A preferred solid oral dosage form of the invention comprises thalidomide, anhydrous lactose, microcrystalline cellulose, polyvinylpyrrolidone, stearic acid, colloidal anhydrous silica, and gelatin. See, e.g., U.S. patent application Ser. No. 10/608,077 filed Jun. 30, 2003, the entirety of which is incorporated herein by reference.

### 4.4.2 Rapid Release Dosage Forms

Single unit dosage forms of the invention can be rapid release dosage forms such as, but not limited to, rapidly dissolving tablets, tapes, transdermal, suspension and liquid dosage forms. The dosage forms provide immediate or rapid release of one or more active agents. For example, rapidly dissolving tablets or tapes can be simply inserted into the mouth of a patient and easily dissolved in oral fluids to achieve a desired therapeutic effect. Rapid release dosage forms of the invention disintegrate rapidly in the mouth to form a suspension of particles and release their contents so as not to interfere with the normal bioavailability of the active ingredient.

Rapid release dosage forms can be prepared by methods of pharmacy well known to those skilled in the art. Examples include, but are not limited to, those described in Remington's Pharmaceutical Sciences, 18th ed., Mack Publishing, Easton Pa. (1990); U.S. Pharmacopoeia No. 23, Chap. 1216 (1995); and U.S. Pat. Nos. 3,962,417, 4,613,497, 4,940,588, 5,055,306, 5,178,878, 5,225,197, 5,464,632, and 6,024,981, each of which is incorporated herein by reference. For example, a coating that rapidly dissolves can be used to permit more rapid release of the active agent(s). The amount of a coating agent and thickness of the coating can vary, depending on the type of formulation, but are readily determined to those of ordinary skill in the art. Where more rapid release of active agent(s) is desired, one skilled in the art would easily recognize the type and thickness of the coating, based on characteristics such as desired blood levels of active agent(s), rate of release, solubility of active agent(s), and desired performance of the dosage form.

### 4.4.3 Delayed Release Dosage Forms

Active agents of the invention can be administered by controlled release means or by delivery devices that are well known to those of ordinary skill in the art. Examples include, but are not limited to, those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active agents using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active agents of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active agent) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active agent can be stimulated by various conditions including, but not limited to, pH, temperature, enzymes, water, or other physiological conditions or compounds.

### 4.4.4 Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active agents disclosed herein can also be incorporated into the parenteral dosage forms of the invention. For example, cyclodextrin and its derivatives can be used to increase the solubility of thalidomide, and its derivatives. See, e.g., U.S. Pat. No. 5,134,127, which is incorporated herein by reference.

### 4.4.5 Topical and Mucosal Dosage Forms

Topical and mucosal dosage forms of the invention include, but are not limited to, sprays, aerosols, solutions, emulsions, suspensions, or other forms known to one of skill in the art. See, e.g., Remington's Pharmaceutical Sciences, 16^{th} and 18^{th} eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are non-toxic and pharmaceutically acceptable. Moisturizers or humectants can also be added to pharmaceutical compositions and dosage forms if desired. Examples of such additional agents are well known in the art. See, e.g., Remington's Pharmaceutical Sciences, 16^{th} and 18^{th} eds., Mack Publishing, Easton Pa. (1980 & 1990).

The pH of a pharmaceutical composition or dosage form may also be adjusted to improve delivery of one or more active agents. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active agents so as to improve delivery. In this regard, stearates can serve as a lipid vehicle for the formulation, as an emulsifying agent or surfactant, and as a delivery-enhancing or penetration-enhancing agent. Different salts, hydrates or solvates of the active agents can be used to further adjust the properties of the resulting composition.

### 4.4.6 Kits

In some cases, active agents of the invention are not administered to a patient at the same time or by the same route of administration. This invention therefore encompasses kits which, when used by the medical practitioner, can simplify the administration of appropriate amounts of active agents to a patient.

A typical kit of the invention comprises a dosage form of thalidomide, or a pharmaceutically acceptable salt, solvate (e.g., hydrate), stereoisomer, prodrug, or clathrate thereof. Kits encompassed by this invention can further comprise additional active agents such as amlodipine, dilitazem, nifedipine, adenosine, epoprostenol (Floran®), treprostinil (Remodulin®), bosentan (Tracleer®), warfarin (Coumadin®), tadalafil (Cialis®), simvastatin (Zocor®), omapatrilat (Vanlev®), irbesartan (Avapro®), pravastatin (Pravachol®), digoxin, nitric oxide, L-arginine, iloprost, betaprost, and sildenafil (Viagra®), or a combination thereof. Examples of the additional active agents include, but are not limited to, those disclosed herein (see, e.g., section 4.2).

Kits of the invention can further comprise devices that are used to administer the active agents. Examples of such devices include, but are not limited to, syringes, drip bags, patches, and inhalers.

Kits of the invention can further comprise pharmaceutically acceptable vehicles that can be used to administer one or more active agents. For example, if an active agent is provided in a solid form that must be reconstituted for parenteral administration, the kit can comprise a sealed container of a suitable vehicle in which the active agent can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 5. EXAMPLES

The following studies are intended to further illustrate the invention without limiting its scope.

### 5.1. Pharmacology Studies

One of the biological effects exerted by thalidomide is the reduction of synthesis of TNF-α. Thalidomide enhance the degradation of TNF-α mRNA. Inhibition of TNF-α production following LPS-stimulation of human PBMC by thalidomide was investigated in vitro. The IC₅₀'s of thalidomide for inhibiting production of TNF-α following LPS-stimulation of PBMC was ˜194 μM (50.1 μg/mL).

### 5.2. Clinical Studies in PH Patients

Clinical Study 1

Thalidomide is administered in an amount of from about 200 to about 800 mg per day to patients with PH for three months. The study is randomized, double-blind and placebo controlled. A total of 20 patients is enrolled, 10 to receive the compound of the invention and 10 to receive placebo. The patients are stable on continuous prostacyclin and have more than 70 mm Hg of pulmonary artery systolic pressure. The patients are dosed at the start of the study with 200 mg, then increased on week 2 and 3 to 400 mg, then a maximum dose 800 mg from week 4 through the duration of the three months. A right heart catherization is performed at baseline and 3 months. Patients are monitored at routine monthly visits. Neurologic examinations are done at baseline, 1, 2 and 3 months. Patients are monitored for sedation and peripheral neuropathy at baseline, 1, 2 and 3 months. ANC is monitored at 1, 2 and 3 months.

Clinical Study 2

In one embodiment of the invention, thalidomide is administered in a single or divided daily doses in an amount of from about 200 to about 800 mg/day. The compound is administered to patients with PH for 12 weeks, who are subsequently evaluated for a decline in walk distance, dyspnea score, functional class, pulmonary hemodynamic response. The first study enrolls 32 patients with PH. Patients are all in modified New York Heart Association functional class III at the onset of the study. Patents are maximally treated and are stable on conventional therapy, including calcium channel antagonists and diuretics. Two thirds of patients receive 400 mg of thalidomide for four weeks followed by 800 mg of the compound for eight weeks. One third of patients receive placebo. The primary efficacy endpoint is a 6-minute walk distance. Patients receiving the compound of the invention walk an average of 70 meters farther after 12 weeks while placebo patients have a decline in walk distance. In addition, the treated patients have improvements in dyspnea score and functional class compared with placebo patients. Pulmonary hemodynamic measurements reveal decreases in pulmonary arterial pressure and pulmonary vascular resistance, and increase in cardiac output after 12 weeks of the treatment, compared with worsening of pulmonary hemodynamics in placebo patients. All these changes in treated patients are highly significant compared with placebo.

Expanded Study

On the basis of the results of the above study 2, the clinical study is expanded with additional 213 PH patients for at least 16 weeks. The study is conducted with patients with PH, WHO functional class III or IV. Two hundred thirteen patients are randomized to receive either 200 mg bid or 400 mg bid of thalidomide or placebo in a 1:1:1 ratio. The primary endpoint, a 6-minute walk distance, is evaluated at 16 weeks. The treated patients walk 36.4 meters further at 16 weeks compared to a 7.8 meter reduction in walk distance in the placebo group, for a treatment effect of 44.2 meters. Clinical worsening, defined by death, premature withdrawal from study, hospitalization for worsening of PH or institution of epoprostenol, occur in 37% of placebo-treated patients, compared with 11% of the patients treated with the compound of the invention. Functional class is improved significantly more in treated patients than placebo patients.

Embodiments of the invention described herein are only a sampling of the scope of the invention. The full scope of the invention is better understood with reference to the attached claims.

1. A method of treating, preventing or managing pulmonary hypertension, which comprises administering to a patient in need of such treatment, prevention or management a therapeutically or prophylactically effective amount of thalidomide, or a pharmaceutically acceptable salt, solvate or stereoisomer thereof.

2. The method of claim 1, which further comprises administering to the patient a therapeutically or prophylactically effective amount of a second active agent.

3. The method of claim 2, wherein the second active agent is capable of reducing pulmonary artery pressure or a symptom of the pulmonary hypertension.

4. The method of claim 2, wherein the second active agent is an anticoagulant, diuretic, cardiac glycoside, calcium channel blocker, vasodilator, prostacyclin analogue, endothelin antagonist, phosphodiesterase inhibitor, endopeptidase inhibitor, lipid lowering agent, or a thromboxane inhibitor.

5. The method of claim 2, wherein the second active agent is amlodipine, diltiazem, nifedipine, epoprostenol, treprostinil, bosentan, warfarin, tadalafil, simvastatin, omapatrilat, irbesartan, pravastatin, digoxin, nitric oxide, L-arginine, iloprost, betaprost, or sildenafil.

6. The method of claim 1, wherein the pulmonary hypertension is primary pulmonary hypertension or secondary pulmonary hypertension.

7. The method of claim 1, wherein the pulmonary hypertension is functional class I, II, III or IV pulmonary hypertension.

8. A method of treating or managing pulmonary hypertension, which comprises administering to a patient in need of such treatment or management a therapeutically or prophylactically effective amount of thalidomide, or a pharmaceutically acceptable salt, solvate or stereoisomer thereof, before, during or after surgery.

9. A method of treating or managing pulmonary hypertension, which comprises administering to a patient in need of such treatment or management a therapeutically or prophylactically effective amount of thalidomide, or a pharmaceutically acceptable salt, solvate or stereoisomer thereof, before, during or after lung transplantation.
